(19) United States
(12) Patent Application Publication
(10) Pub. No.: US 2024/0395405 A1
(43) Pub. Date: Nov. 28, 2024

(54) MACHINE LEARNING MODELS FOR ESTIMATION OF LUNG ALVEOLAR VENTILATION PERFUSION MISMATCH

(71) Applicant: META FLOW LTD., Tel Aviv (IL)

(72) Inventors: Merav MOR, Tel-Aviv (IL); Michal MOR, Tel-Aviv (IL); Avi SMILA, Tel-Aviv (IL); Daniel TAL, Tel-Aviv (IL); Dror CEDER, Herzliya (IL)

(73) Assignee: META FLOW LTD., Tel Aviv (IL)

(21) Appl. No.: 18/693,175

(22) PCT Filed: Sep. 21, 2022
(86) PCT No.: PCT/IL2022/051008

§ 371 Date: Mar. 19, 2024

Related U.S. Application Data
(60) Provisional application No. 63/341,006, filed on May 12, 2022.
(60) Provisional application No. 63/246,807, filed on Sep. 22, 2021.

(51) Int. Cl. G16H 50/20; A61B 5/00; A61B 5/083; G06N 20/00

(52) U.S. Cl. CPC G16H 50/20; A61B 5/0836; A61B 5/7246; A61B 5/7267; G06N 20/00

(57) ABSTRACT

There is provided a computer-implemented method of training a machine learning model for computing an estimate of a ventilation inhomogeneity parameter indicating a ventilation inhomogeneity state of a subject, comprising: creating a training dataset comprising a plurality of records, each record including a plurality of measurements of a single breath maneuver of a respective subject measured by at least one sensor including at least one CO₂ sensor and at least one pressure sensor, labelled with a ground truth label of the ventilation inhomogeneity parameter indicating the ventilation inhomogeneity state, and training a machine learning model on the training dataset for generating an outcome of an estimate of a target ventilation inhomogeneity parameter for a target subject in response to an input of a target plurality of measurements of a single breath maneuver of the subject sensed by the at least one sensor.

### RELATED APPLICATION(S)

This application claims the benefit of priority of U.S. Provisional Patent Application No. 63/246,807 filed on Sep. 22, 2021 and U.S. Provisional Patent Application 63/341,006 filed on May 12, 2022, the contents of which are incorporated herein by reference in their entirety.

### FIELD AND BACKGROUND OF THE INVENTION

The present invention, in some embodiments thereof, relates to respiratory parameter measurements and, more specifically, but not exclusively, to methods of volumetric capnographic measurements.

Ventilation perfusion mismatch (V′/Q′) defects are defects in the total lung ventilation/perfusion ratio (V′/Q′ ratio). In a healthy individual with healthy lungs and healthy circulatory system, the rate of alveolar ventilation to the rate of pulmonary blood flow is in balance, to maintain oxygen transportation from the lung (i.e., ventilation) into the blood stream (i.e., circulation/perfusion). A V′/Q′ mismatch indicates that areas of the lungs receive oxygen but not enough blood flow, or the lungs receive enough blood flow but not enough oxygen.

### SUMMARY OF THE INVENTION

According to a first aspect, a computer-implemented method for training a machine learning model for computing an estimate of a ventilation inhomogeneity parameter indicating a ventilation inhomogeneity state of a subject, comprises: creating a training dataset comprising a plurality of records, each record including a plurality of measurements of a single breath maneuver of a respective subject measured by at least one sensor including at least one CO₂ sensor and at least one pressure sensor, labelled with a ground truth label of the ventilation inhomogeneity parameter indicating the ventilation inhomogeneity state, and training a machine learning model on the training dataset for generating an outcome of an estimate of a target ventilation inhomogeneity parameter for a target subject in response to an input of a target plurality of measurements of a single breath maneuver of the subject sensed by the at least one sensor.

According to a second aspect, a computer-implemented method of computing an estimate of a ventilation inhomogeneity parameter of the subject, comprises: feeding a plurality of measurements obtained during a single breath maneuver of the subject, sensed by at least one sensor, into a machine learning model trained on a training dataset of a plurality of records of a plurality of sample individuals, each record including a respective plurality of measurements of a respective single breath maneuver measured by the at least one sensor, each respective record labeled with a ground truth of the ventilation inhomogeneity parameter indicating a ventilation inhomogeneity state of the respective sample individual, and obtaining, as an outcome of the machine learning model, an indication of the ventilation inhomogeneity state for the subject.

According to a third aspect, a device for computing an estimate of a ventilation inhomogeneity parameter of the subject, comprises: at least one hardware processor executing a code for: feeding a plurality of measurements of a single breath maneuver of the subject, sensed by at least one sensor including at least one CO₂ sensor and at least one pressure sensor, into a machine learning model trained on a training dataset of a plurality of records of a plurality of sample individuals, each record including a respective plurality of measurements of a respective single breath maneuver of measured by the at least one sensor, each respective record labeled with a ground truth of the ventilation inhomogeneity parameter indicating a ventilation inhomogeneity state of the respective sample individual, and obtaining, as an outcome of the machine learning model, an estimate of the ventilation inhomogeneity parameter for the subject.

In a further implementation form of the first, second, and third aspects, the indication of the ventilation inhomogeneity parameter comprises a value indicating a Ventilation-Perfusion ratio (V′/Q′).

In a further implementation form of the first, second, and third aspects, the V′/Q′ ratio is obtained by a member selected from a group comprising: MIGET, radionuclide imaging, and MRI with intravenous contrast.

In a further implementation form of the first, second, and third aspects, further comprising computing a correlation function that correlates between the ventilation inhomogeneity parameter and a member selected from a group consisting of: V′/Q′, dead space to tidal volume ratio (VD/VT), and minute ventilation/carbon dioxide production (V′E/V′CO₂), by computing a mapping between the ground truth ventilation inhomogeneity parameters obtained for a plurality of sample individuals, and the respective member of the group.

In a further implementation form of the first, second, and third aspects, further comprising computing a correlation function that correlates between the ventilation inhomogeneity parameter and at least one medical parameter of the subject, by computing a mapping between the ground truth ventilation inhomogeneity parameters obtained for a plurality of sample individuals, and at least one medical parameter obtained for each of the plurality of sample individuals.

In a further implementation form of the first, second, and third aspects, the medical parameter is selected from a group comprising: an indication of congestive heart failure (CHF), an indication of circulatory failure, an indication of diffusion impairment, an indication of right to left shunt, and an indication of pulmonary hypertension.

In a further implementation form of the first, second, and third aspects, the ventilation inhomogeneity parameter comprises a value selected from a group consisting of: indicating dead space to tidal volume ratio (VD/VT), and indicating minute ventilation/carbon dioxide production (V′E/V′CO₂).

In a further implementation form of the first, second, and third aspects, the ventilation inhomogeneity parameter comprises a value indicating a Ventilation-Perfusion ratio (V′/Q′).

In a further implementation form of the first, second, and third aspects, the ventilation inhomogeneity parameter comprises a value selected from a group consisting of: indicating dead space to tidal volume ratio (VD/VT), and indicating minute ventilation/carbon dioxide production (V′E/V′CO₂).

In a further implementation form of the first, second, and third aspects, at least one sensor includes a combined carbon dioxide sensor and a pressure sensor.

In a further implementation form of the first, second, and third aspects, at least one sensor excludes an oxygen sensor.

In a further implementation form of the first, second, and third aspects, the at least one sensor excludes a flow sensor.

In a further implementation form of the first, second, and third aspects, further comprising generating instructions and presentation on a display and/or audible feedback on speakers, instructing the subject to perform the single breath maneuver by instructing the subject to inhale during an inhalation phase, a holding phase during which the instructions are for the subject hold the inhaled air, and an exhalation phase during which the instructions are for the subject to exhale the air held during the holding phase.

In a further implementation form of the first, second, and third aspects, the plurality of measurements obtained during the single breath maneuver includes at least measurements of CO₂ sensed by a CO₂ sensor during the exhaling phase that are normalized using measurement of at least a pressure sensor and CO₂ sensed by the CO₂ sensor obtained during the inhale and hold.

In a further implementation form of the first, second, and third aspects, further comprising accessing at least one subject parameter of the subject, wherein feeding comprises feeding the at least one subject parameter and the plurality of measurements into the machine learning model, wherein each record of the training dataset further includes a respective at least one subject parameter of the respective subject.

In a further implementation form of the first, second, and third aspects, the at least one subject parameter is selected from a group consisting of: height, gender, age, ambient temperature, ambient pressure, geographical location, and ventilation inhomogeneity state selected from a group comprising of pre/post-workout, pre/post-meal, the morning after wake-up, and before bedtime.

In a further implementation form of the first, second, and third aspects, further comprising accessing at least one additional measurement of the subject made from at least one additional sensor, wherein feeding comprises feeding the at least one additional measurement and the plurality of measurements into the machine learning model, wherein each record of the training dataset further includes a respective at least one additional measurement of the respective subject made from the at least one additional sensor and/or external data source.

In a further implementation form of the first, second, and third aspects, the at least one additional measurement is selected from a group consisting of: blood pressure, heart rate, heart rate variability (HRV), blood glucose, high density lipoprotein (HDL)-cholesterol, triglyceride level (TG), body composition, ketosis levels, breathing rate (BR), hemoglobin concentration (Hb), oxyhemoglobin concentration (HbO₂), oxygen saturation (SpO₂)), Body Fat Percentage (% BF), waist circumference, Waist-to-hip ratio, heart rate, and visceral fat.

In a further implementation form of the first, second, and third aspects, further comprising: analyzing the estimate of the ventilation inhomogeneity parameter for the subject according to a set of rules, and generating instruction for presentation on a display and/or playing on speakers for treating the subject based on the analysis and continuous monitoring according to at least one defined goal and/or target.

In a further implementation form of the first, second, and third aspects, the at least one sensor comprises a wearable printed flexible sensor.

In a further implementation form of the first, second, and third aspects, further comprising computing a member of a group consisting of: V′/Q′, VD/VT, and V′E/V′CO₂, according to a precomputed correlation function.

In a further implementation form of the first, second, and third aspects, further comprising the at least one sensor including the at least one CO₂ sensor and the at least one pressure sensor.

In a further implementation form of the first, second, and third aspects, further comprising code for: creating the training dataset comprising the plurality of records, and training the machine learning model on the training dataset.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a block diagram of a system for computing an estimate of ventilation inhomogeneity parameters of a subject indicating a ventilation inhomogeneity state of the subject using a machine learning model, and/or for training the machine learning model, in accordance with embodiments of the present invention;

FIG. 2 which is a flowchart of a method for computing an estimate of ventilation inhomogeneity parameters of a subject indicating a ventilation inhomogeneity state of the subject using a machine learning model, in accordance with some embodiments of the present invention; and

FIG. 3 is a flowchart of a method for training a machine learning model for generating an estimate of a ventilation inhomogeneity parameter of a subject indicating a ventilation inhomogeneity state of the subject, in accordance with some embodiments of the present invention.

### DESCRIPTION OF SPECIFIC EMBODIMENTS OF THE INVENTION

The present invention, in some embodiments thereof, relates to ventilation inhomogeneity parameter measurements and, more specifically, but not exclusively, to systems and methods for volumetric capnography-based ventilation inhomogeneity parameter determination.

As used herein, V′=volume flow=volume/time=ventilation=liters/min (as an example, or other units may be used).

As used herein, Q′=blood flow=volume blood/time=circulation=liters/min (as an example, or other units may be used).

As used herein, the term “estimate” and “indication”, of the ventilation inhomogeneity parameter(s) are interchangeable.

As used herein, the term “subject” and “individual” are used interchangeably.

As used herein, the term “ventilation inhomogeneity parameter” refers to a parameter that provides an indication of an amount of ventilation inhomogeneity (or inhomogeneous ventilation) in a subject. Ventilation inhomogeneity refers to a reduction in the amount of oxygen reaching the lungs, and/or to a reduction in the amount of blood reaching the lungs (for oxygen-carbon dioxide gas exchange), for example, in comparison to normal healthy lungs, ventilation inhomogeneity may be measured using a V′/Q′ parameter that indicates a ratio between ventilation (i.e., oxygen reaching the lungs) and perfusion (i.e., blood reaching the lungs). In healthy lungs, the ventilation should be approximately matched to perfusion, i.e., enough blood is provided to the lungs and/or enough air is provided to the lungs to perform oxygen-carbon dioxide exchange between the blood and lungs. V′/Q′ values below and/or above one or more thresholds indicating a metabolic state (e.g., 0.8, 0.9, 1, 1.1, 1.2, or other values) indicating a problem with insufficient air reaching the lung and/or insufficient blood reaching the lungs.

An aspect of some embodiments of the present invention relates to system, methods, apparatus (i.e., a computing device), and/or code instructions (stored on a memory and executable by one or more hardware processors) for estimating a ventilation inhomogeneity parameter indicating a ventilation inhomogeneity state of a subject from measurements made during a single breath maneuver. The single breath maneuver includes a single inhale phase, breath-holding phase and a single exhale phase, and/or other breathing maneuverer(s). The measurements may be made by a carbon dioxide (CO₂) sensor and a pressure sensor, optionally a combination of CO₂ and pressure sensor. The sensors may exclude an oxygen sensor, i.e., in some implementations no oxygen data is used. The sensors may exclude a flow sensor, i.e., in some implementations no flow data is used. The measurements are fed into a machine learning (ML) model, for example, through a process embedded in the firmware (FW) and/or application software. An estimate of the ventilation inhomogeneity parameter indicating ventilation inhomogeneity state is obtained as an outcome of the ML model. The ventilation inhomogeneity parameter may be a value indicative of ventilation-perfusion (V′/Q′) ratio. The ventilation inhomogeneity parameter may indicate a degree of V′/Q′ mismatch, for example, when the ventilation inhomogeneity parameter indicating V′/Q′ ratio is above and/or below one or more thresholds. The ventilation inhomogeneity parameter may be expressed as an estimate of the ventilation to perfusion ratio (V′/Q′). Alternatively, or additionally, the ventilation inhomogeneity parameter may be correlated (e.g., by a correlation function and/or other correlation ML model) to the estimate of V′/Q′. The V′/Q′ represents a golden standard measurement. It is noted that other measurements, which may represent golden standards, may be used. Alternatively or additionally, the ventilation inhomogeneity parameter (e.g., indicating V′/Q′) may be correlated (e.g., by a correlation function and/or other correlation ML model) to a medical parameter indicating a medical condition in which V′/Q′ mismatches are observed, for example, congestive heart failure (CHF), circulatory failure, diffusion impairment, right to left shunt, an indication of other cardiac problems, and an indication of pulmonary hypertension.

An aspect of some embodiments of the present invention relates to system, methods, apparatus (i.e., computing device), and/or code instructions (stored on a memory and executable by one or more hardware processors) for training a ML model to generate an outcome of an estimate of ventilation inhomogeneity parameter(s) (e.g., V′/Q′) indicating a ventilation inhomogeneity state of a subject in response to an input of sensor measurements obtained during a single breath maneuver. A training dataset of multiple records is created, where each record includes measurements made during a single breath maneuver of a respective sample individual, labeled as baseline of the ventilation inhomogeneity state. Records may further include other data, as described herein. The ML model is trained on the training dataset.

Optionally, the ground truth (baseline) of the ventilation inhomogeneity parameter is computed using one or more equations. A correlation function between the ventilation inhomogeneity parameter and V′/Q′ (e.g., measured using standard conditions) may be computed. The ventilation inhomogeneity parameter outcome of the ML model may be correlated with the V′/Q′. Alternatively, the ground truth (baseline) of the ventilation inhomogeneity parameter is V′/Q′, which may be measured using standard methodology.

At least some implementations of the systems, methods, apparatus, and/or code instructions described herein address the technical challenges of improving a user's experience of measuring ventilation inhomogeneity parameter(s), optionally mismatches in V′/Q′ and/or medical parameters that are correlated with mismatches in V′/Q′ (e.g., CHF, pulmonary hypertension, circulatory failure, diffusion impairment, right to left shunt and the like).

Some technical problems of standard existing approaches for measuring ventilation inhomogeneity and/or mismatches in V′/Q′ are now described. At least some embodiments described herein are designed to improve over the standard existing approaches, as described herein. In healthy subjects at rest, PACO₂ can be estimated from end-tidal carbon dioxide pressure (PETCO₂) or from arterial carbon dioxide pressure (PaCO₂) by substituting PACO₂ with PaCO₂ in the Bohr equation. However, this has effects on the dead space estimation, especially when the calculation is used in subjects with inhomogeneous ventilation. Calculation of dead space is referred to as physiologic dead space and is not equal to the anatomic dead space, as it includes unventilated alveolar space influencing the PaCO₂. The additional alveolar dead space comprises un-perfused alveoli, wasted ventilation (V′), and resulting perfusion (Q′) heterogeneity. With an increased V′/Q′ ratio, for example, in circulatory failure, diffusion impairment, and right to left shunt.

At least some implementations of the systems, methods, apparatus, and/or code instructions described herein address the technical challenges of improving a user's experience of measuring medical parameters, for example, an indication of CHF, circulatory failure, diffusion impairment, right to left shunt, and pulmonary hypertension. Existing approaches of measuring medical parameters are now discussed. Numerous scientific studies have demonstrated the relationship between resting PETCO₂ and cardiac output. PETCO₂ obtained during exercise has also been correlated with cardiac output in patients with heart failure and can reflect disease severity in this population. Resting and peak exercise PETCO₂, as well as the highest increase from rest during a progressive cardiopulmonary exercise test (CPX), demonstrate independent prognostic value in patients with heart failure. In patients with pulmonary hypertension, resting tidal volume (VT), and peak exercise PETCO₂ are significantly correlated with pulmonary arterial pressures and can thus provide a noninvasive reflection of disease severity. CPX is used in the detection of exercise-induced right-to-left shunting. With gas exchange measures and resting echocardiogram as the reference. The sensitivity, specificity, positive and negative predictive values, and accuracy have been reported to be between 90% and 96%: (1) An abrupt and sustained increase in P_{ET}O₂ with a simultaneous sustained decrease in P_{ET}CO₂ (2) an abrupt and sustained increase in the RER and (3) an associated decline in oxygen pulse. The technical problem is that measuring medical parameters using standard approaches is difficult, time consuming, requires specialized equipment, requires specially trained personnel, and is conducted in a specialized lab setting. At least some implementations of the systems, methods, apparatus, and/or code instructions described herein, based on the ML model described herein, enable measuring the medical parameters using an approach that is technically simpler for the user, can be done by the user themselves without necessarily requiring specially trained personnel, can be done in many places and does not require a specialized setting, as described herein.

At least some implementations of the systems, methods, apparatus, and/or code instructions described herein address the technical challenges described herein, and/or improve the technology described herein, for example, by providing one or more of: a portable device that enables individual users to perform their own ventilation inhomogeneity parameter measurements (and/or medical parameter measurement) without requiring standard non-portable and/or complex specialized equipment, simplifying the process for the user to perform ventilation inhomogeneity parameter measurements, and/or enabling measuring ventilation inhomogeneity parameter(s) in a resting state.

At least some implementations of the systems, methods, apparatus, and/or code instructions described herein improve the technology of measuring ventilation inhomogeneity parameter(s) of users, by one or more of providing a device and/or method that is based on a single breath pattern (e.g., inhale, hold, exhale), measuring ventilation inhomogeneity parameter(s) during resting state, and simplifying the design and/or method without requiring oxygen sensor(s).

At least some implementations of the systems, methods, apparatus, and/or code instructions described herein provide a technical solution to the above-mentioned technical challenges and/or improve the technical solution, by one or more of:
A trained machine learning model that generates an outcome of a ventilation inhomogeneity parameter indicating an estimate of a ventilation inhomogeneity state of a subject in response to measurements obtained during a single breath maneuver. The ventilation inhomogeneity parameter may be estimated using measurements obtained during the single breath maneuver, which may be done “on the go” and/or during a non-resting state, in contrast to standard measurement approaches that require complex equipment, a specialized lab setting, and/or trained personnel.
A carbon dioxide (CO₂) and pressure sensor (optionally as a combined single sensing device) that obtains the measurement signals during the single breath maneuver, optionally excluding the use of an oxygen sensor. No oxygen sensor is required, which may simplify the design of the testing device in which the sensors are installed, and may reduce the complexity of the sensor design and/or may improve the computational efficiency of a processor and/or memory by reducing the data that is processed.
Additional data that may be used to train the ML model and/or fed into the ML in combination with the measurement made by the sensor(s). Examples of additional data are described herein.
Estimate of V′/Q′ and/or other ventilation inhomogeneity measurements are obtained based on the sensor measurements obtained during a single breath maneuver. The V′/Q′ and/or other ventilation inhomogeneity parameters are obtained, from the single breath maneuver, optionally without using oxygen sensors, compared to standard approaches that estimate V′/Q′ and/or other ventilation inhomogeneity measurements using complex equipment which requires many breathing samples.
Using ventilation inhomogeneity parameters as indicators to determine medical parameters, for treating and/or preventing medical conditions in the subject, such as CHF, circulatory failure, diffusion impairment, right to left shunt, pulmonary hypertension, metabolic syndrome, type 2 diabetes, obesity, cardiovascular disease, stress, recovery, or poor sleep conditions. Since the ventilation inhomogeneity parameter is easily measured, at many time points during a day the ventilation inhomogeneity parameter may be obtained for the subject, throughout the day and/or at different physiological states, for example, fasting, post exercise, and post meal. The medical parameter(s) may be fed into other applications that generate instructions for the subject on how to adjust lifestyle to prevent or treat the medical conditions based on the input of the physiological parameter(s).

One example of a ventilation inhomogeneity parameter that may be estimated using measurements obtained by sensor(s) during the single breath maneuver that are fed into the ML model, as described herein in at least some embodiments, is V′/Q′-mismatch. The V′/Q′ ratio is the amount of air that reaches the alveoli divided by the amount of blood flow in the capillaries of the lungs. A V′/Q′ mismatch happens when part of the lung receives oxygen without enough blood flow, or blood flow without enough oxygen delivery. Such situation may arise, for example, in obstructed airways, or capillary blood vessels obstructed by blood clots. Such situation may arise in medical conditions that allow bringing in the air (ventilation) but impede on extracting oxygen from that air. In another example, bringing in blood but not enough oxygen is delivered.

A V′/Q′ mismatch can cause hypoxemia, which is low oxygen levels in blood. Not having enough blood oxygen can lead to respiratory failure. A V′/Q′ mismatch is often seen in obesity or chronic diseases such as pre-diabetes, type 2 diabetes mellitus, cardiovascular diseases, or metabolic syndrome and is considered a key determinant of cardio metabolic health.

At least some embodiments described herein improve over standard approaches for measuring ventilation inhomogeneity, optionally for measuring a V′/Q′ ratio which may be a mismatch, by providing a simpler approach, as described herein in additional detail. Examples of standard approaches include functional techniques such as MIGET and the three-compartment model. The MIGET (Multiple Inert Gas Elimination Technique) uses six different dissolved gases infused intravenously. The arteriovenous difference in gas concentration and the known blood:

gas partition coefficient of each gas is used to determine the distribution of V′/Q′. The Three-compartment model assumes there are only three gas exchange units: one completely composed circulation, one completely composed of dead space, and one which has both (shunt). This technique requires the measurement of PaO₂, PaCO₂ and the estimation of alveolar O₂ and CO₂ partial pressures. Other standard imaging techniques include radionuclide imaging such as SPECT V′/Q′ scans, PET scans, and MRI using intravenous contrast such as gadolinium (Ga), He or Xe. Such traditional approaches are invasive (requiring blood sampling), and/or require specialized equipment operated by trained professionals in a hospital setting. In contrast, some implementations described herein enable a user to measure ventilation inhomogeneity and/or V′/Q′ mismatch, outside of the laboratory or hospital settings, without necessarily complex and specialized equipment and/or trained professionals.

Another example of ventilation inhomogeneity parameters that may be estimated through measurements obtained with the single breath maneuver fed into the ML model is the dead space to tidal volume ratio (VD/VT). VD/VT dead space is the volume of gas in the airways and lungs that participates in tidal breathing but does not participate in gas exchange. Examples are the volume of the endotracheal tube or ventilator circuit (apparatus dead space) and the volume of the trachea and central airways (anatomic dead space). A less obvious but still important source of impairment in critically ill patients is alveolar dead space (physiologic dead space). This is dead space from lung units in which ventilation greatly exceeds perfusion. Gas exchange in these over ventilated, under-perfused lung units, is inefficient and abnormal. Either cause of increased VD/VT can cause a decrease in alveolar volume (VA) and hence in alveolar ventilation (V′A). When the cause of increased VD/VT is a lung disease, ventilatory adaptation contributes to keeping VA and PaCO₂ normal.

At least some implementations described herein enable accurate and/or reliable measurement of a subject's VD/VT.

Another example of a ventilation inhomogeneity parameter that may be estimated by feeding measurements obtained during the single breath maneuver into the ML model is the minute ventilation/carbon dioxide production (V′E/V′CO₂) slope. (V′E/V′CO₂) slope has been widely demonstrated to have strong prognostic value in patients with congestive heart failure (CHF). The minute ventilation/carbon dioxide production (V′E/V′CO₂) slope reflects the increase in ventilation in response to CO₂ production, and thus reflects increased ventilatory drive. Changes in the V′E/V′CO₂ slope may be induced by increased number of chemoreceptors, the peripheral receptor response, the ventilatory dead-space, and also by the active muscles engaged in exercise.

At least some implementations described herein enable accurate and/or reliable measurement of a subject's V′E/V′CO₂).

The standard measurement approaches for measuring at least some values described herein, for example, using cardiopulmonary exercise testing (CPET) and other techniques, like echocardiography, are expensive, complex, and/or require trained professionals to operate, and therefore are difficult to use at a person's home and/or in a non-laboratory setting. In contrast, some implementations described herein provide a simple, convenient, user-friendly, and/or cost-effective cardiorespiratory parameter measurement ability, for measuring (V′E/V′CO₂) (e.g., slope) and/or for measuring (V′E/V′CO₂) (e.g., slope) with sensors used to measure the breath carbon dioxide concentrations combined with the breath flow rate, as described herein. At least some applications described hereinafter operate without an oxygen sensor, in contrast to at least some standard approaches. Moreover, the standard measurement devices are bulky, expensive, and require trained staff to operate, for calibration, and maintenance of the equipment. As such, the standard classical devices are suitable only for use in healthcare-, hospital- and laboratory settings. In contrast, at least some applications described herein after enable measuring ventilation inhomogeneity parameters in non-laboratory settings, such as free living conditions, for example, in general practitioner's offices, in health care and weight-loss institutes, gyms and fitness areas, as well as for application by any individual with interest in ventilation inhomogeneity assessment to perform their own measurements.

At least some implementations of the systems, methods, apparatus, and/or code instructions described herein are improvements over prior approaches, for example, as described with reference to US 2016/0220147, by the same Applicant as the present disclosure, incorporated herein by reference in its entirety:
Some prior approaches, such as US 2016/0220147, are based on the user breathing at a target rate (volume per unit time) defined by a target breathing profile. In contrast, in at least some implementations described herein, the subject's breathing is not required to match to a target rate and is not defined by a target breathing profile. The user may breathe at their own rate, at any volume per unit time. This allows the ventilation inhomogeneity parameter to be computed under different and/or dynamic breathing rates, such as during physical activity. The ability to compute the ventilation inhomogeneity parameter for the same person under different breathing rates (e.g., dynamically) is enabled by the ML model, which is trained on a training dataset of different sensor measurements from different subjects made for different breathing rates. It is noted that instructing the subject to perform the single breath maneuver, of breath-hold-exhale, as described herein, does not restrict the user to any target rate.
Some prior approaches, such as US 2016/0220147, require a flow meter to measure the volume per unit time, in order to verify whether the user is breathing according to the target rate defined by the target breathing profile, or not. In contrast, at least some implementations described herein do not necessarily require a flow meter. CO₂ and/or pressure measurements (and/or other measurements) may be sampled along the phases of the single breath (as described herein) and fed into the ML model for obtaining the ventilation inhomogeneity parameter without using flow measurements by a flow meter. Eliminating the need for a flow sensor improves the computational efficiency of the device (e.g., by reducing the amount of data being processed), improves the simplicity of the device (e.g., fewer sensors required), and/or improve the experience of the user (e.g., enabling the user to breath at their own rate rather than follow a specific target breathing rate).

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random-access memory (RAM), a read-only memory (ROM), an erasable programmable read-only memory (EPROM or Flash memory), a static random-access memory (SRAM), a portable compact disc read-only memory (CD-ROM), a digital versatile disk (DVD), a memory stick, a floppy disk, and any suitable combination of the foregoing. A computer-readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer-readable program instructions described herein can be downloaded to respective computing/processing devices from a computer-readable storage medium or to an external computer or external storage device via a network, for example, the Internet, a local area network, a wide area network, and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine-dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

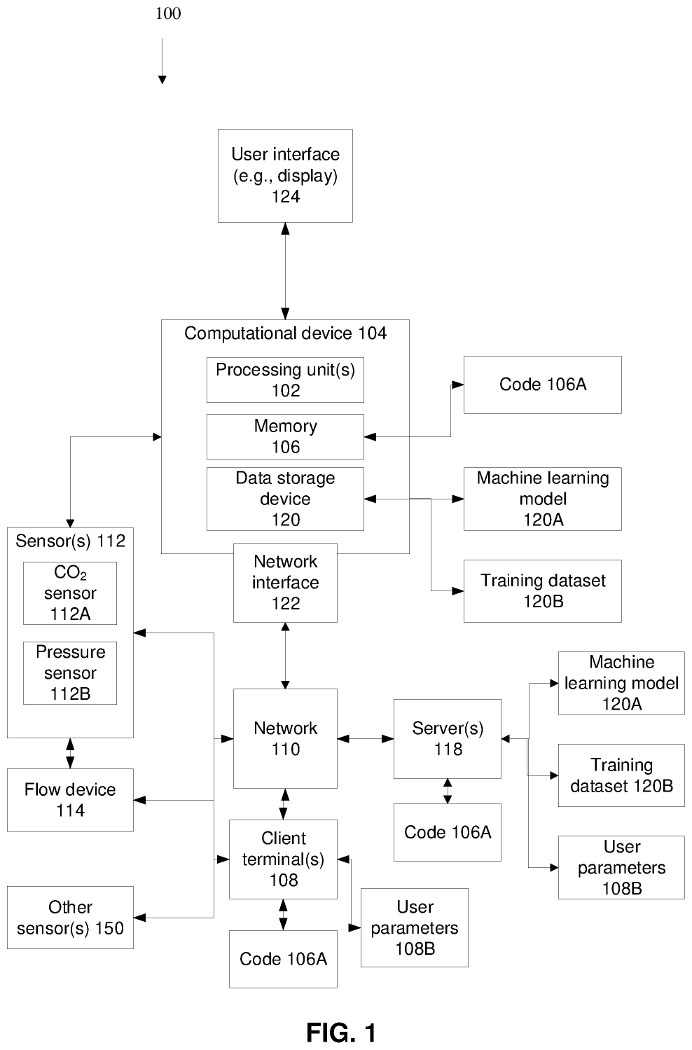

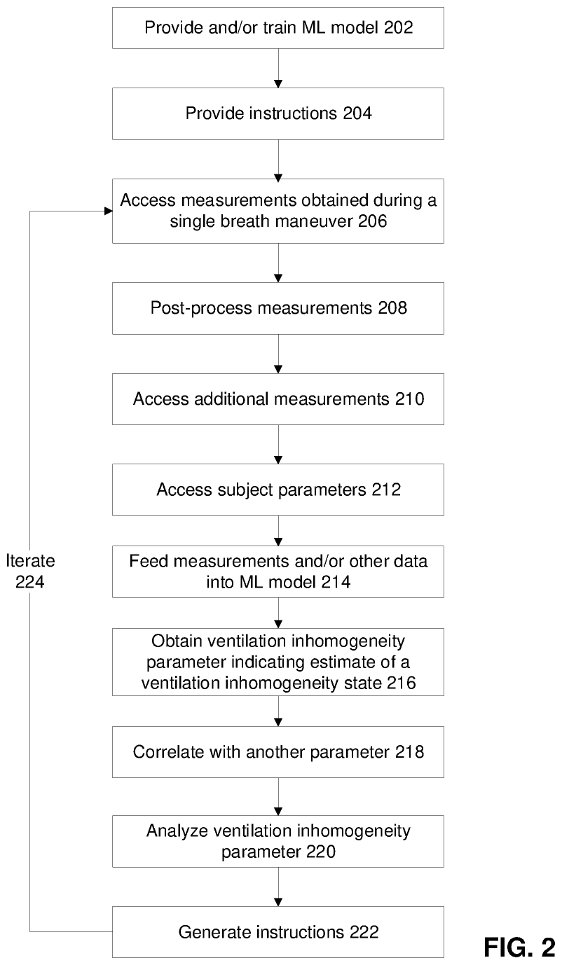

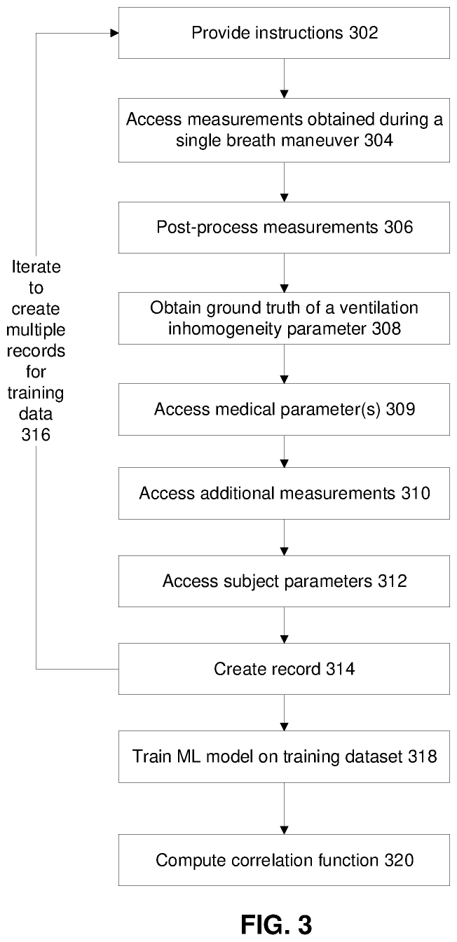

Reference is made to FIG. 1, which is a block diagram of a system 100 for computing an estimate of a ventilation inhomogeneity parameter of a subject indicating a ventilation inhomogeneity state of the subject using a trained machine learning model, and/or for training the machine learning model, in accordance with some embodiments of the present invention. Reference is also made to FIG. 2, which is a flowchart of a method for computing an estimate of a ventilation inhomogeneity parameter of a subject indicating a ventilation inhomogeneity state of the subject using a trained machine learning model, in accordance with some embodiments of the present invention. Reference is also made to FIG. 3, which is a flowchart of a method for training a machine learning model for generating an estimate of a ventilation inhomogeneity parameter of a subject indicating a ventilation inhomogeneity state of the subject, in accordance with some embodiments of the present invention.

System 100 may implement the features of the method described with reference to FIGS. 2-3, by one or more hardware processors 102 of a computing device 104 executing code instructions 106A stored in a memory (also referred to as a program store) 106.

Computing device 104 receives data from sensors 112 associated with a flow device 114. Exemplary sensors include a CO₂ sensor 112A and a pressure sensor 112B. In some implementations, an O₂ sensor is excluded from sensor(s) 112, i.e., no O₂ sensors is used, and/or no O₂ measurements are obtained by computing device 104. Alternatively, in other implementations, the O₂ sensor is included in sensor(s) 112, i.e., O₂ sensors are used, and/or O₂ measurements are obtained by computing device 104. In some implementations, a flow sensor is excluded from sensor(s) 112, i.e., no flow sensors is used, and/or no flow measurements are obtained by computing device 104.

Pressure sensor 112B may be used to perform pressure compensation in CO₂ nondispersive infrared (NDIR) sensor measurements, in order to compensate for the difference between the actual measurements of barometric pressure and sea level.

Flow device 114 is designed to house sensors 112 for measuring air being inhaled by a user and/or air exhaled by a user. An exemplary flow device 114 is described, for example, with reference to US20200116632, incorporated herein by reference in its entirety.

Computing device 104 may receive data from other sensor(s) 150, for example, a heart rate sensor (e.g., heart rate and/or heart rate variability), respiration sensor (e.g., breathing rate), acetone sensor (e.g., acetone levels), and the like, as described herein.

Computing device 104 may receive and/or store subject parameters 108B, for example, demographic parameters, medical history, medical parameters (e.g., medical measurements), geographic location, etc., as described herein. User parameters 108B may be, for example, manually entered by a user, stored on a local data storage device of computing device 104, stored on a local data stored device of a client terminal of the user and provided to computing device 104 over a network 110, and/or remotely stored on another server.

Computing device 104 feeds the input into ML model 120A to obtain the ventilation inhomogeneity parameter, as described herein.

Multiple architectures of system 100 based on computing device 104 may be implemented. In an exemplary implementation, computing device 104 may be implemented as a component within flow device 114, for example, as a card and/or circuitry installed within the housing of flow device 114. In another implementation, computing device 104 may be an external device that is in local communication with flow device 104, for example, computing device 104 is a mobile device (e.g., smartphone, laptop, watch computed) connected to flow device 114, for example, by a cable (e.g., USB) and/or short-range wireless connection. In such implementation, each computing device 104 may be associated with a single or small number of flow devices, for example, a user uses their own smartphone to connect to their own flow device. In yet another implementation, computing device 104 may be implemented as one or more servers (e.g., network server, web server, a computing cloud, a virtual server) that provides services to multiple flow devices 114, for example, providing centralized services to remotely located flow devices. Flow devices may directly communicate with computing device 104 acting as the server over network 110, and/or may indirectly communicate with the server using an intermediary device, such as a client terminal (e.g., mobile device) that locally communicates with flow device 114 and remotely communicates with the server over network 110.

It is noted that different components of system 100 may reside on different devices such as computing device 104, server(s) 118, and client terminal(s) 108, for example, sensor 112, code 106A, user parameter(s) 108B, ML model 120 and/or training dataset 120B, and others, may be located on different devices in different architectures.

In another exemplary architecture of system 100, sensor(s) 112 are implemented within computing device 104, for example, as a device into which the user breaths for example, as described with reference to US 2020/0116632, incorporated herein by reference in its entirety. Client terminal 108 may be implemented as a smartphone (or other mobile device) which runs code 106A (e.g., downloaded from server(s) 118) such as an App. Measurement data sensed by sensor(s) 112 of computing device 104 may be forwarded by client terminal 108 over network to server(s) 118. Computing device 104 and/or client terminal 108 may perform local data pre-processing, such as formatting. Server(s) 118 may feed the sensor measurements into ML model 120A, which may be trained on training dataset 120B locally and/or on yet another server. Server(s) 118 may receive sensor measurements from multiple different client terminals 108, i.e., providing centralized services.

In yet another architecture, sensor(s) 112 may be connected to client terminal 108. In yet another architecture, one or more sensor 112 are connected to client terminal 108, such as pressure sensor 112B sensing ambient pressure, and/or one or more other sensors 112 are connected to computing device 104 such as CO₂ sensor 112A.

Computing device 104 may be implemented as, for example, a client terminal, a server, a virtual machine, a virtual server, a computing cloud, a mobile device, a desktop computer, a thin client, a Smartphone, a Tablet computer, a laptop computer, a wearable computer, glasses computer, and a watch computer.

Hardware processor(s) 102 may be implemented, for example, as a central processing unit(s) (CPU), a graphics processing unit(s) (GPU), field programmable gate array(s) (FPGA), digital signal processor(s) (DSP), and application specific integrated circuit(s) (ASIC). Processor(s) 102 may include one or more processors (homogenous or heterogeneous), which may be arranged for parallel processing, as clusters and/or as one or more multi core processing units.

Memory 106 stores code instructions executable by hardware processor(s) 102. Exemplary memories 106 include a random-access memory (RAM), read-only memory (ROM), a storage device, non-volatile memory, magnetic media, semiconductor memory devices, hard drive, removable storage, and optical media (e.g., DVD, CD-ROM). For example, memory 106 may store code 106A that execute one or more acts of the method described with reference to FIGS. 2-3.

Computing device 104 (and/or server(s) 118) may include a data storage device 122 for storing data, for example, machine learning model 120A, and/or training dataset 120B. Data storage device 122 may be implemented as, for example, a memory, a local hard-drive, a removable storage device, an optical disk, a storage device, and/or as a remote server and/or computing cloud (e.g., accessed over network 110). It is noted that code 120A-B may be stored in data storage device 122, with executing portions loaded into memory 106 for execution by processor(s) 102.

Machine learning model 120A may be implemented, for example, as a classifier, a statistical classifier, one or more neural networks of various architectures (e.g., convolutional, fully connected, deep, encoder-decoder, recurrent, graph, combination of multiple architectures), support vector machines (SVM), logistic regression, k-nearest neighbor, decision trees, boosting, random forest, a regressor and the like. ML model may be trained using supervised approaches and/or unsupervised approaches on training dataset 120B.

Computing device 104 may include a network interface 122, for connecting to network 110, for example, one or more of, a wire connection (e.g., physical port), a wireless connection (e.g., antenna), a network interface card, a wireless interface to connect to a wireless network, a physical interface for connecting to a cable for network connectivity, and/or virtual interfaces (e.g., software interface, application programming interface (API), software development kit (SDK), virtual network connection, a virtual interface implemented in software, network communication software providing higher layers of network connectivity).

Network 110 may be implemented as, for example, the internet, a local area network, a virtual network, a wireless network, a cellular network, a local bus, a point-to-point link (e.g., wired), and/or combinations of the aforementioned.

Computing device 104 may communicate with one or more of the following over networks 110:
Flow device 114 to obtain measurements made by sensors 112 and optionally from Other Sensors(s) 150
Client terminal 108, for example, when computing device 104 is implemented as a server in communication with multiple client terminals 108 each connected to a respective flow device 114. Measurements made by sensors 112, and optionally from other sensors(s) 150, of each respective flow device 114 may be provided to computing device 104 via locally connected client terminals 108, and/or directly to the computational device 104 internal communication interfaces, for example UART, I2C, TWI, SPI. User parameters 108B of respective users may be locally stored on respective client terminals 108 and/or entered by users via respective client terminals, and provided to computing device 114 and/or Server(s) 118.
Server(s) 118, for example, to obtain updated versions of ML model 120A. It is noted that training of ML model 120A may be performed by computing device 104, or remotely by server 118, where trained ML model 120A is provided to computing device 104.

Computing device 104 may include and/or be in communication with one or more physical user interfaces 124 that include provide a mechanism to enter data (e.g., user parameters 108B) and/or view data (e.g., the computed ventilation inhomogeneity parameter, and/or instructions for treatment of the subject based on an analysis of the ventilation inhomogeneity parameter) for example, one or more of, a touchscreen, a display, gesture activation devices, a keyboard, a mouse, and voice activated software using speakers and microphone.

Referring now back to FIG. 2, at 202, a machine learning model(s) is trained and/or provided. An exemplary approach of training the machine learning model is described, for example, with reference to FIG. 3.

Different machine learning models may be trained and/or provided and/or selected, optionally according to a correlation between available data for the current user and data used to train the respective machine learning model. For example, a user may select a model that receives blood pressure data from a blood pressure sensor and/or heart rate variability data from a heart rate sensor. In another example, a model used for weight loss may be selected. In yet another example, a model that does not need O₂ input is may be selected.

Models may be selected, for example, manually by a user (e.g., using a user interface), automatically by code (e.g., according to an analysis of connected sensors and/or accessible data), and/or predefined (e.g., access to certain selected model granted to the current user).

At 204, instructions may be provided to the user.

Instructions may be presented on a display (e.g., text, images, videos, animations) and/or audible feedback is played on speakers.

The instructions may be for instructing the user to perform a single breath maneuver. The single breath maneuver includes an inhalation phase in which instructions are for the subject to inhale, a holding phase during which the instructions are for the subject to hold the inhaled air, and an exhalation phase during which the instructions are for the subject to exhale the air held during the holding phase.

Instructions may be provided before the user performs any breath maneuvers, during the breath maneuvers (e.g., synchronized with the breath maneuvers such as by detecting the phase of the single breath maneuver by analyzing data from the sensor(s)) and providing the instructions according to the current detected phase), and/or after the breath maneuvers.

The instructions may be presented in response to user input, for example, the user pressing an icon, pressing a button, and/or speaking indicating readiness for the instructions. The instructions may be dynamically presented in response to sensing the phase of the user, for example, by analyzing sensor data to determine which phase the user is performing, and instructing the user during the phase and/or instructing the next phase. For example, sensing breath pressure drops, and/or changes in carbon dioxide concentration to determine the current phase.

The instructions may be for the user to enter a resting state, for example, sit for 15 minutes after the workout.

At 206, measurements obtained during the single breath maneuver of the subject are accessed.

The measurements obtained during the single breath maneuver are determined by one or more sensors. The sensor(s) include at least a carbon dioxide (CO₂) sensor(s) and a pressure sensor. Optionally, a single combined sensor performing both the CO₂ detection and the pressure measurement.

In some implementations, the sensor(s) exclude an oxygen (O₂) sensor. It is noted that an oxygen sensor is used in standard approaches of measuring ventilation, for example, as described herein. Performing measurements without oxygen sensors may improve the design of the measurement device (which is simpler since no oxygen sensor is used), improve computational efficiency of processors performing computations (since oxygen data is not used) and/or reduces cost of the device (since no oxygen sensor, which may be expensive, is used).

Measurements may be obtained by sensors installed within a device, for example, as described with reference to US 2020/0116632, incorporated herein by reference in its entirety. The sensors perform measurements while the user inhales and/or exhales into the device as part of performing the single breath maneuver.

Sensors may include a wearable printed flexible sensor and/or printable calorimetric sensor(s).

Optionally, measurements are obtained during a complete single-breath maneuver. Measurement samples may be obtained at a certain rate, for example, 1-10, or 3-5, or about minimum. 3 samples per second (Hz), or other values. Optionally, a minimal number of samples are obtained for analysis, for example, at least about 10, or 20, or 30, or 50, or intermediate or higher values.

The measurements may be obtained during a resting state when the subject is resting, for example, has been sitting in a chair and/or lying down. The physiological state of the subject, such as resting and/or non-resting combined with sub-metrics (e.g., waist circumference, waist-to-hip ratio, blood pressure, heart rate, heart rate variability (HRV), blood glucose, visceral fat, high density lipoprotein (HDL)-cholesterol, triglycerides (TG)) may be provided as input which is fed into the machine learning model, as described herein. The sub-metrics may be obtained, for example, from other sensor(s), from a health record of the subject, from manual input using a user interface, and the like.

At 208, post-processing of the measurements may be performed.

Measurements obtained from the exhalation phase of the single breath maneuver may be normalized using measurement information obtained during the inhale phase and/or breath holding phase. The normalization may be done per subject breath maneuver and/or per testing device and/or per sensor(s), using the measurements obtained from the subject and/or by the testing device and/or by the sensor(s). The normalization enables comparing data from different users and/or different sensors and/or different testing devices, such that feeding the normalized data into the ML model, and/or training the ML model using normalized data.

Alternatively, or additionally, measurements may be filtered, using low and/or high pass threshold filtering, and/or statistical polynomial fitting, and/or other Bayesian based filtering, and/or other Bayesian based filtering.

The phase, i.e., inhalation, hold, exhale, when respective measurements are obtained may be determined, for example, based on an analysis of the CO₂ and/or pressure sensor signals, and/or other sensors. For example, a low CO₂ level indicates the inhale phase, constant and maintained (e.g., within a range) pressure may indicate the holding phase, and a high CO₂ level may indicate the exhalation phase.

At 210, one or more additional measurements of the subject made from one or more additional sensors may be accessed. The additional measurements may be made during one or more phases of the single breath maneuver, optionally in parallel with the measurements made by the sensors described with reference to 206, such as the CO₂ and/or pressure sensors. The additional measurements may be made before and/or after the single breath maneuver, such as measurements unrelated to the single breath maneuver. The additional measurements may be made during a non-resting state, and/or during the resting state. The additional measurements may define the non-resting state, for example, the amount of activity during the non-resting state. One or more values may be computed from one or more measurements of one or more sensors.

Optionally, heart rate and/or heart rate variability are measured by a heart rate sensor. It is noted that heart rate variability may be computed by analyzing the heart rate measurements obtained by the heart rate sensor. Alternatively, or additionally, a heart rate variability sensor is used to measure heart rate variability. The heart rate and/or heart rate variability may be measured during the non-resting state and/or resting state. The heart rate and/or heart rate variability may define the amount of activity during the non-resting state, for example, a faster heart rate may indicate more intense activity such as running, while a lower (but above resting) heart rate may indicate less intense activity such as walking. The heart rate may be used, for example, to normalize measurements obtained during the resting state for comparison with measurements obtained during the non-resting state, where the heart rate during the resting state may indicate a baseline.

Examples of additional measurement(s) obtained from other sensors and/or computed from measurements obtained by other sensors(s) include biomarkers, blood glucose, body composition, ketosis levels, breathing rate (BR), hemoglobin concentration (Hb), oxyhemoglobin concentration (HbO₂), oxygen saturation (SpO₂), Body Fat Percentage (% BF) and Body Composition.

At 212, one or more subject parameters of the subject may be accessed. Subject parameters may be obtained, for example, by manual input from a user (e.g., via a user interface), from a local storage device, and/or from a remote storage cloud and/or server. Subject parameters may be represented, for example, as metadata tags, and/or as values of defined fields.

The subject parameters may include, for example, one or more of:
Demographic parameters, such as age and gender.
Measurements of the subject, such as height and weight.
Parameters of the environment of where the subject is located, for example, ambient temperature, ambient pressure, and geographical location,
Metrics of the subject, for example, e.g., waist circumference, waist-to-hip ratio, blood pressure, heart rate, HRV, hypertension, blood glucose, visceral fat, HDL-cholesterol, TG.
Medical history, for example, medical conditions, (e.g., pre-diabetes, type 2 diabetes, cardiovascular disease), alcohol consumption, prescribed medications, and smoking history.

It is noted that some values may be referred to herein as subject parameters and/or additional sensor measurements.

At 214, the measurements (e.g., obtained as described with reference to 206, and/or the post-processed measurements as described with reference to 208) are fed into the machine learning model.

The measurements may be fed as a combination with additional measurements made by other sensor(s) (e.g., heart rate, heart rate variability, ketosis level, breathing rate, and the like as described herein) and/or subject parameter(s), as described herein.

At 216, the ventilation inhomogeneity parameter, indicating an estimate of the ventilation inhomogeneity state for the subject, is obtained as an outcome of the machine learning model.

Optionally, the estimate of the ventilation inhomogeneity state comprises an estimate of the Ventilation/Perfusion ratio (V′/Q′). The estimated V′/Q′ may be obtained using measurements, such as CO₂ and/or pressure measurements, obtained during a single breath maneuver, in contrast to standard approaches for obtaining V′/Q′. Standard approaches for computing the ventilation inhomogeneity parameter and/or the V′/Q′ value may be used during the training phase to obtain ground truth ventilation inhomogeneity parameter and/or V′/Q′ labels for records (e.g., as described herein with reference to FIG. 3).

Other examples of ventilation inhomogeneity parameters include VD/VT (dead space volume/tidal volume), and V′E/V′CO₂ (Minute Ventilation/V′CO₂). The VD/VT and/or V′E/V′CO₂ are discussed in detail herein, for example, above.

Alternatively, or additionally, the estimate of the ventilation inhomogeneity parameter for the subject is a single score. The single score may be evaluated on its own, for example, analyzed, such as to detect a trend. The single score may be correlated with other ventilation inhomogeneity values, such as V′/Q′ and/or others described herein.

At 218, one or more correlations may be computed.

The correlation may be computed when the estimate of the ventilation inhomogeneity parameter for the subject (obtained as an outcome of the ML model) is a single score.

Optionally, the ventilation inhomogeneity parameter (e.g., single score), for example V′/Q′, is correlated with one or more medical parameters. Exemplary medical parameters include an indication of congestive heart failure (CHF), an indication of circulatory failure, an indication of diffusion impairment, an indication of right to left shunt, and an indication of pulmonary hypertension.

Alternatively or additionally, the ventilation inhomogeneity parameter represents a unique measurement. The ventilation inhomogeneity parameter may be correlated with other values, for example, one or more of: V′/Q′, dead space to tidal volume ratio (VD/VT), and minute ventilation/carbon dioxide production (V′E/V′CO₂).

The correlation may be computed using a predefined correlation function and/or trained correlation ML model. The predefined correlation function may be computed, for example, as described with reference to 320 of FIG. 3.

Optionally, the estimate of the ventilation inhomogeneity for the subject is based on a predefined statistical agreement method with ground truth measurement of the medical parameter(s), ground truth V′/Q′ measurements, and other values described herein. The statistical agreement may be computed by computing a mapping between (e.g., single) scores computed for multiple sample individuals from respective measurements of the single breath maneuver measured by the sensor(s), and the ground truth measurements of the medical parameters and/or ground truth V′/Q′ measurements.

The predefined correlation function and/or computed mapping may be implemented as one or more implementations of ML models described herein.

Alternatively or additionally to performing the correlation using the ventilation inhomogeneity parameter obtained from the ML model, the ML model is trained on a training dataset of multiple records, where a record includes measurements made by the sensor(s) and ground truth labels indicating the medical parameter(s) which may be measured using best clinical practices for measuring indications of medical parameter(s), and/or ground truth labels indicating V′/Q′ and other values described herein. Such ML model may directly generate the medical parameter and/or V′/Q′ as an outcome in response to an input of measurements made by the sensor(s) in addition to, and/or as an alternative to, first obtaining the ventilation inhomogeneity parameter as an outcome of the ML model and then performing the correlation to obtain the medical parameter(s) and/or V′/Q′. It is noted however that the ventilation inhomogeneity parameter is useful, for example, to help explore different reasons for a V′/Q′ mismatch, and/or to track changes in V′/Q′, in addition to and/or as an alternative to tracking medical parameter(s).

At 220, the estimate of the ventilation inhomogeneity parameter and/or the medical parameter for the subject may be analyzed.

The analysis may be performed according to a set of rules, for example, to determine whether the set of rules is met or not. For example, the ventilation inhomogeneity parameter and/or the medical parameter may be evaluated against a threshold, to determine whether the respective parameter is above and/or below one or more defined thresholds. For example, when the parameter comprises a V′/Q′ mismatch, the threshold may be above 0.8 or 0.9, where the V′/Q′ value is too low, or too high.

The analysis may be performed by feeding the estimate of the ventilation inhomogeneity parameter and/or the medical parameter and/or the V′/Q′ parameter into another executing process, for example, an application designed to treat and/or prevent conditions (e.g., generate instructions for treatment of and/or prevention of conditions), for example, V′/Q′ mismatch, increased VD/VT which indicate respiratory failure (e.g., acute respiratory distress syndrome (ARDS)) and high V′E/V′CO₂ slope which may indicate cardiovascular vascular disorders (e.g., congestive heart failure (CHF)). The process may be locally running, and/or running on another device such as a remote server. Examples of processes include “Fat Loss” programs, “Stress” relief applications, Sleep Quality improvement applications, Female Monthly Cycle tracking and lifestyle improvement programs connected to Health Insurance providers and/or wellness and health programs, and other wellness devices.

It is noted that the estimate of the ventilation inhomogeneity parameter and/or the medical parameter(s) and/or V′Q′ are not necessarily obtained from an integrated sensor, but could be web-based data sources. Moreover, the analysis approach described herein is not necessarily limited to the measurement hardware device described herein.

At 222, instructions for treating the subject and/or for preventing medical condition(s) may be generated based on the estimate of the ventilation inhomogeneity parameter and/or based on the medical parameter(s) and/or based on V′/Q′. The instructions may be presented on a display (e.g., text, image, video) and/or played on speakers.

The instructions may be based on continuous monitoring of the subject, optionally based on defined goals and/or targets. For example, indicating to the subject whether the goal and/or target has been reached, and if not, indicating to the subject what to do in order to increase likelihood of reaching the goal and/or target. Feedback to the subject may be provided, for example, using another App installed on a mobile device of the user, and/or by email messages, and the like.

It is noted that at 224, one or more features described with reference to 206-222 may be iterated. Iterations may be performed, for example, at different times per day or once every few days, in conjunction with evaluating different work and/or fat loss programs. Iterations may be performed for monitoring the ventilation inhomogeneity parameter and/or medical parameter(s) over time, for example, to detect an increase, decrease, and/or stability. Predictions of future value(s) of the ventilation inhomogeneity parameter(s) and/or future values of the medical parameter(s) may be computed, for example, using historical values, such as by regression and/or other approaches. The predictions of the future value(s) may be compared to a set of rules, such as a threshold, which may trigger instructions for the user. For example, when the V′/Q′ of the user is being monitored, and remains stable at around 0.9, and is predicted to decrease to below 0.8 in about a week, a message may be generated indicating the user to consult their doctor, and/or the message may instruct the user on actions to take to avoid the V′/Q′ from decreasing.

Referring back to FIG. 3, at 302, instructions are presented to the respective sample individual, i.e., current user. The instructions may explain how to perform the single breath maneuver. The instructions may be to enter resting physiological state information. Additional examples of instructions are described with reference to 204 of FIG. 2.

At 304, measurements made by one or more sensors during the single breath maneuver of the respective subject are accessed. The measurements may be made by CO₂ sensor(s) and/or pressure sensor(s), optionally a combination thereof, optionally excluding oxygen sensors. Additional details of obtaining measurements are described, for example, with reference to 206 of FIG. 2.

At 306, post-processing of the measurements may be performed. Measurements obtained during the exhaling phase of the single breath maneuver may be normalized using measurement obtained during the inhalation phase and/or holding phase. Normalization may be per measurements obtained per subject. Additional details are described, for example, with reference to 208 of FIG. 2.

At 308, a ground truth of a ventilation inhomogeneity parameter indicating the ventilation inhomogeneity state of the respective sample individual is obtained. The ground truth may be a single score, which is computed using one or more equations from the measurements made by one or more sensors.

Alternatively, or additionally, the ground truth may be a Ventilation Perfusion ratio (V′/Q′). The V′/Q′ may be obtained by computing a ratio of an amount of carbon dioxide produced from ventilation, sensed by the carbon dioxide sensor and pressure sensor, over multiple breaths while the respective sample individual is at rest. The ground truth ventilation inhomogeneity parameter (e.g., V′/Q′) may be obtained, for example, using standard approaches such as MIGET and the three-compartment model, radionuclide imaging PET scans, MRI using intravenous contrast, and a standard V′/Q′ scan, as described herein. Alternatively, or additionally, the ground truth may be of other exemplary ventilation inhomogeneity values, such as VD/VT, and V′E/V′CO₂, as described herein.

The ground truth values may be measured in parallel with the measurements made during the single breath maneuver. The ground truth values may be measured independently of the measurements made during the single breath maneuver, for example, made before or after the singe breathe maneuver.

At 309, one or more medical parameter(s) of the subject may be obtained. The medical parameters may be values indicating a state of a medical condition of the subject, for example, an indication of CHF, an indication of circulatory failure, an indication of diffusion impairment, an indication of right to left shunt, and an indication of pulmonary hypertension. For example, an indication of left ventricular ejection fraction (LVEF) which provides an indication of CHF, and a mean pulmonary artery pressure which is an indication of pulmonary hypertension. The medical parameters may be measured using standard best practice clinical protocols.

The medical parameter(s) may be measured in parallel with the measurements made during the single breath maneuver. The medical parameter(s) may be measured independently of the measurements made during the single breath maneuver, for example, made before or after the singe breath maneuver.

At 310, one or more additional measurements obtained by other sensor(s) and/or computed from measurements obtained by other sensors(s) are accessed. The additional measurements may be made in parallel with the measurements made during the single breath maneuver. The additional measurement may be made independently of the measurements made during the single breath maneuver.

Examples of additional measurements include heart rate and/or heart variability made by a heart rate sensor. Other examples of additional measurements obtained from other sensors and/or computed from measurements obtained by other sensors(s) include blood pressure, HRV, blood glucose, HDL-cholesterol, TG, body composition, ketosis levels, breathing rate (BR), hemoglobin concentration (Hb), oxyhemoglobin concentration (HbO₂), oxygen saturation (SpO₂), Body Fat Percentage (% BF), and body composition.

Additional details of obtaining additional measurements are described, for example, with reference to 210 of FIG. 2.

At 312, one or more subject parameters of the respective subject are accessed, for example, Waist circumference, Waist-to-hip ratio, an indication of Associated diseases (such as cardiovascular disease, type II diabetes), and demographic parameters. Additional details of obtaining subject parameters are described, for example, with reference to 212 of FIG. 2.

At 314, a record is created. The record is for the respective sample individual, and includes the measurements obtained during the single breath maneuver (e.g., as described with reference to 304), and may include the additional sensor measurements (e.g., as described with reference to 310), and/or may include the subject parameters (e.g., as described with reference to 312) and/or may include the medical parameter(s) (e.g., as described with reference to 309). The record is labeled with a ground truth indication of the ventilation inhomogeneity parameter (e.g., as described with reference to 308). Alternatively or additionally, record is labeled with a ground truth indication of the medical parameter(s).

At 316, features described with reference to 302-314 may be iterated, optionally per sample individual, and/or multiple times for each sample individual, to obtain multiple records. A training dataset that includes the multiple records is created.

At 318, a machine learning model is trained on the training dataset. The generated ML model generates an outcome of an estimate of a target ventilation inhomogeneity parameter for a target subject in response to an input of target measurements of a single breath maneuver of the subject sensed by sensor(s), optionally carbon dioxide and pressure, optionally excluding an oxygen sensor.

At 320, a correlation function and/or other correlation ML model may be computed and/or trained. The correlation function and/or correlation ML model is computed and/or trained for mapping between ventilation inhomogeneity values (e.g., represented as single scores) V′/Q′ values (and/or other ventilation inhomogeneity parameters), and one or more of: medical parameter(s), V′/Q′, VD/VT, and V′E/V′CO₂. The correlation function and/or ML model is trained and/or computed on a correlation dataset of records, where each record includes a respective ventilation inhomogeneity value (e.g., represented as a single score) for the subject computed from measurements of sensors (e.g., CO₂ and/or pressure) obtained during the single breath maneuver, and a ground truth of one or more of: medical parameter(s), V′/Q′ value(s), VD/VT, and V′E/V′CO₂, which may be measured using standard approaches such as V′/Q′ scan and other approaches described herein. The correlated estimated medical parameter and/or V′/Q′ (and/or other values) is obtained by the correlation function without requiring that the user undergo a V′/Q′ scan and/or other standard measurement approaches, but rather using the measurements obtained during the single breath maneuver, as described herein.

Some not necessarily limiting examples (e.g., use cases) of applications of embodiments described herein and/or as claimed are now described. The different applications may be implemented using embodiments described herein, for example, instructions are generated as described with reference to 222 of FIG. 2 according to the application being implemented.

Examples described below relate to VD/VT, and/or V′E/V′CO₂, measured and monitored using devices and/or methods described herein, is a concept developed as a result of better understanding how the ventilation inhomogeneity works at a molecular level. This concept is directly connected to developing better Respiratory and Cardiovascular health. Use case of a ventilation inhomogeneity parameter measured by devices and/or methods described herein, include cases that result in prevention and monitoring of possible respiratory (e.g., ARDS) and Cardiovascular disorders (e.g., CHF).

For Example: The phase III slope of expired CO₂ flattens and plateaus. This phase represents the pure alveolar gas compartment that exists once CO₂ from the airway-alveolar interface is washed out. Increase of III slope is an index of V′/Q′ mismatching, a decrease in phase III slope corresponds to improved V′/Q′ homogeneity.

Additional Interest in the measurement of dead space in acute respiratory distress syndrome (ARDS) patients is based on the study by Nuckton and colleagues, who demonstrated elevated Enghoff physiological dead space is a strong independent predictor of mortality in early phase ARDS.

It is expected that during the life of a patent maturing from this application many relevant machine learning models will be developed and the scope of the term machine learning model is intended to include all such new technologies a priori.

As used herein the term “about” refers to ±10%.

The terms “comprise”, “comprising”, “includes”, “including”, “having” and their conjugates mean “including but not limited to”. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1-25. (canceled)

26. A computer-implemented method for estimating a parameter indicating a ventilation state of a subject, via an input-record of the subject; wherein the input-record comprises data extracted from measurements of a single breath maneuver of the subject, measured by sensors comprising: at least one sensor CO₂ sensor and at least one pressure sensor, the method comprising:
creating a training dataset comprising:
a plurality of training input-records, each associated with a respective training-subject; and
associated plurality of ground truth labels each indicating the ventilation state of their associated training-subject;
training a machine learning model (MLM) via the training dataset;
applying said trained MLM on the input-record of the subject, to obtain the estimated parameter, indicating the ventilation state of the subject.

27. The method of claim 26, wherein the parameter comprises a value indicating a metabolic property.

28. The method of claim 27, wherein the metabolic property is at least one selected from: Rest Metabolic rate (RMR), Respiratory Energy Expenditure (REE), Respiratory Quotient (RQ) and Oxygen consumption, Respiratory Exchange Ratio (RER).

29. The method of claim 26, wherein the estimated parameter comprises a value indicating a Ventilation-Perfusion ratio (V′/Q′).

30. The method of claim 26, wherein the ground truth labels are obtained via at least one selected from: MIGET, radionuclide imaging, MRI with intravenous contrast, CO₂ capnography, and diffusing capacity of the lungs for carbon monoxide (DLCO), metabolic cart.

31. The method of claim 26, wherein the extracted data comprises raw data of the measurements.

32. The method of claim 26, wherein the extracted data comprises dead space to tidal volume ratio (VD/VT), extracted from the raw data of the measurements.

33. The method of claim 26, wherein the extracted data comprises a slope of exhaled CO₂ vs. flow, extracted from the raw data of the measurements.

34. The method of claim 33, wherein the slope is extracted at phase III.

35. The method of claim 26, wherein the extracted data comprises a slope of exhaled CO₂ vs. volume, extracted from the raw data of the measurements.

36. The method of claim 35, wherein the slope is extracted at phase III.

37. The method of claim 26, wherein the extracted data comprises a slope of exhaled minute ventilation/CO₂ production, extracted from the raw data of the measurements.

38. The method of claim 37, wherein the slope is extracted at phase III.

39. The method of claim 26, wherein the extracted data comprises exhaled CO₂ percentage, at end of phase III, vs. time and/or vs. volume, extracted from the raw data of the measurements.

40. The method of claim 26, further comprising correlating between the parameter and a medical indication for at least one selected from: congestive heart failure (CHF), circulatory failure, diffusion impairment, gas exchange efficiency, right to left shunt, pulmonary hypertension, metabolic syndrome, type 2 diabetes, obesity, cardiovascular disease, stress, recovery, poor sleep condition, acute respiratory distress syndrome (ARDS).

41. The method of claim 26, further comprising generating instructions for the subject to perform the single breath maneuver and presenting said instructions via a display- and/or audible-device; wherein said instructions composing:
an inhalation phase, instructing the subject to inhale for a predetermined inhale-time and profile;
a holding phase, instructing to the subject to hold the inhaled air for a predetermined hold-time; and
an exhalation phase, instructing the subject to exhale the held air for a predetermined exhale-time and profile.

42. The method of claim 41, further comprising normalizing the measurements of the CO₂ exhalation phase, using measurements obtained during the inhalation and/or holding phases selected from: pressure, CO₂, volume, and any combination thereof.

43. The method of claim 26, wherein at least one of the following holds true:
the sensors exclude at least one of: an oxygen sensor, and a flow sensor;
the input-record comprises only non-invasive data;
the input-record further comprises at least one additional measurement selected from: blood pressure, heart rate, heart rate variability (HRV), blood glucose, high density lipoprotein (HDL)-cholesterol, triglyceride level (TG), body composition, ketosis levels, breathing rate (BR), hemoglobin concentration (Hb), oxyhemoglobin concentration (HbO₂), oxygen saturation (SpO₂), body fat percentage (% BF), waist circumference, waist-to-hip ratio, heart rate, and visceral fat.
the input-record further comprises at least one data-element selected from:
subject's height;
subject's gender;
subject's age;
room's ambient temperature;
room's ambient pressure;
geographical location; and
subject's at least one status selected from: pre/post-workout, pre/post-meal, and after wake-up/before-bedtime;
the method, further comprising:
analyzing the estimated parameter and comparing to a predetermined threshold, and
generating instruction to present recommendations via a display and/speakers for treating the subject, based on the analysis;
the analysis and recommendation are based on periodic estimation of said parameter, and predetermined goals and/or target.

44. A device configured to estimate a parameter indicating a ventilation state of a subject, via an input-record of the subject; wherein the input-record comprises data extracted from measurements of a single breath maneuver of the subject, measured by sensors comprising: at least one sensor CO₂ sensor and at least one pressure sensor, the device comprising:
at least one processor configured to implement the method steps according to claim 26; and
at least one input-device, configured to receive, at least part of the input record;
at least one output-device, configured to present, at least the estimated parameter.

45. A computer code configured for executing the method according to claim 26.
